(19) United States
(12) Patent Application Publication
(10) Pub. No.: US 2021/0375153 A1
(43) Pub. Date: Dec. 2, 2021

(54) TRAINING THE NERVOUS SYSTEM THROUGH NEURO-CHEMICAL AND NEURO-CIRCUITRY CONDITIONING APPARATUS FOR VR VISION, AND RELATED SYSTEM

(71) Applicants: Faryt Kalhil, Sunrise, FL (US); Juan Carlos Abello, Miami, FL (US)

(72) Inventors: Faryt Kalhil, Sunrise, FL (US); Juan Carlos Abello, Miami, FL (US)

(21) Appl. No.: 17/334,257

(22) Filed: May 28, 2021

Related U.S. Application Data
(60) Provisional application No. 63/032,559, filed on May 30, 2020.

(51) Int. Cl. G09B 19/00; G06T 15/00; G06F 3/01; G09B 9/00

(52) U.S. Cl. CPC G09B 19/00; G09B 9/00; G06F 3/011; G06T 15/00

(57) ABSTRACT

A computer-implemented neuro-chemical and neuro-circuitry conditioning training method that employs specially programmed computing devices and systems that guides a user through a process to discover, extract, describe, and synthesize their inner dream (or dreams) and interacts with a user in order to design their dream as they have imagined it. Through the assets, environment, and other such deliverables generated from the various method modules along with specially programed computing devices, the user is enabled to interact with the assets and the generated environment and replay those interactions on-demand thereby effecting neuro-chemical and neuro-circuitry training.

### CROSS-REFERENCE TO RELATED APPLICATIONS

This application is a non-provisional of, and claims priority to, U.S. Provisional Application Ser. No. 63/032,559, filed on May 30, 2020; which is incorporated by reference herein.

### FIELD OF THE INVENTION

The present invention generally relates to training our nervous system and our subconscious mind through the use of a program and tools for conditioning our neuro-chemistry and neuro-circuitry and more specifically relates to a method of neuro-chemical and neuro-circuitry conditioning training that employs a specially programmed visualization apparatus and related system.

### BACKGROUND OF THE INVENTION

### Description of the State of the Art

Mental and nervous system conditioning via neuro-chemical and neuro-circuitry conditioning training methods is known to realign thought patterns by using the imagination to reinforce positive thoughts and actions, or to diminish negative thoughts and actions. In the art, positive reinforcement is known to strengthen neural pathways and create new synaptic connections when a person continuously visualizes a desired outcome. The old adage applies, “before you can achieve it, first you must believe it.” We are taught to visualize our dreams by imagining our perfect picture of success. It has been demonstrated in the art that those who constantly visualize their goals or dreams are those who often achieve them. However, we are not given the tools to actually immerse into a world of vivid imagination where we can clearly see ourselves living our dream.

For example, it is known that to utilize vision boards with pictures of a house or car or amount of money in the bank, as a constant visual cue and reminder of the desired outcome, helps us elevate our vibrational frequency and begin to attract that which we desire. Also, there are audio programs that allow listeners to meditate and try to visualize the dream or objective. Regardless of the external tools employed for the neuro-chemical and neuro-circuitry conditioning training, most people have a hard time being able to visualize clearly, internalize and truly “live” the dream, since the images in their minds are not often vivid, lacking much reality. This ends up resulting in weak visualization at best and a lack of clarity or affinity with the dream. A weak or under-defined physical stimulus—such as a visualization—is not conducive to strong or efficient neuro-chemical and neuro-circuitry conditioning. The brain just does not “believe” in the stimulus enough to really change. The results are usually poor and often leads to frustration or giving up on the dream.

There is a need, therefore, for an effective, repeatable and easy to use method and tool that allows the common person to overcome the above-stated shortcomings of the known art of visualization to manifest their desires or dreams.

### SUMMARY OF THE INVENTION

Briefly, according to one embodiment of the present disclosure, we disclose a computer-implemented neuro-chemical and neuro-circuitry conditioning training method that employs specially programmed computing devices and systems and includes the steps or acts of: providing an audio-visual program module that guides a user through a process to discover, extract, describe, and synthesize their inner dream (or dreams); providing a specially-programmed computing device that utilizes a user-interface of an intake module to interact with a user in order to design their dream as they have imagined it; capturing by the intake module of user inputs; receiving by a generator module the user inputs from the intake module; generating by the generator module one or more immersion models, or assets, that will be combined into an environment and used in the neural-conditioning phases of the method; compiling by an environment module the one or more immersion models into an environment; providing a hardware system comprising a processor, a storage, and a display, the processor programmed to retrieve from the storage the environment compiled by the environment module; whereby the assets, environment, and other such deliverables from the various method modules are then combined with specially-programed computing devices such that the user can interact with the assets and the generated environment and replay those interactions on-demand.

The invention disclosed herein comprises structured mechanisms and processes to enable the user to first discover, then extract and describe their true inner dream(s). Then, that dream will be experienced by the user via total-immersion conditioning. Examples of such total-immersion conditioning includes experiences created in virtual reality (“VR”) so that with the use of specially-programmed hardware, such as VR goggles or headsets, the user will actually be able to see himself or herself living the dream, in great detail, in true color, with the most advanced Virtual Reality “immersion reality”, and will be able to live and relive this experience using the inventive apparatus, system and method on demand. The repetition of this process will create a new vibration frequency for the user that starts to attract the people, events and circumstances to manifest that reality. Repeated conditioning of situations and outcomes has been shown to create new, and strengthen existing, neural pathways in the brain. It is known in the art, for example, that repeated and continuous practice of mindfulness meditation actually results in the creation of new neural pathways in the brain. Thus, if you can see it in your mind, you can hold it in your hand. Implementing the method, apparatus, and system of the present invention, it is not left to the user's own imaginative abilities or skills—which are often lacking. Rather, it is augmented with the latest virtual reality technology—such as, but not limited to: HD, Ultra-VR with added sensory stimulators, added realism through the added environment that includes family members, friends, etc. . . . —and the most sophisticated neuro-science tools and techniques—such as, but not limited to: tools and techniques that teach your brain how to pay attention to your subconscious thoughts by becoming conscious of your subconscious thoughts, techniques that allow you to decide which thoughts you need to develop and make habitual to become the person you want to be and attract the future you want to create, mental exercises that help you develop the habit of focusing on what you want, instead of what you don't want, and techniques to put yourself in the desired state where you actually feel the emotions and feelings of achieving your dream ahead of the experience, just to name a few.

A revenue model is included as part of the invention. Revenue is derived through, for example, a subscription model where a user may pay for a level of service and, in return, receive guidance, hardware, and custom-programmed visualization tools. The software and computing aspects of the invention may be realized as a custom, or localized, installation or, alternatively, in a software-as-a-service model where the user interacts with the various aspects of the invention via a wide area network, such as the Internet.

### BRIEF DESCRIPTION OF THE DRAWINGS

In the accompanying figures, like reference numerals refer to identical or functionally similar elements throughout the separate views. The accompanying figures, together with the detailed description below are incorporated in and form part of the specification and serve to further illustrate various embodiments and to explain various principles and advantages all in accordance with the present invention, in which:

FIG. 1 is a high-level block diagram of a system, according to an embodiment;

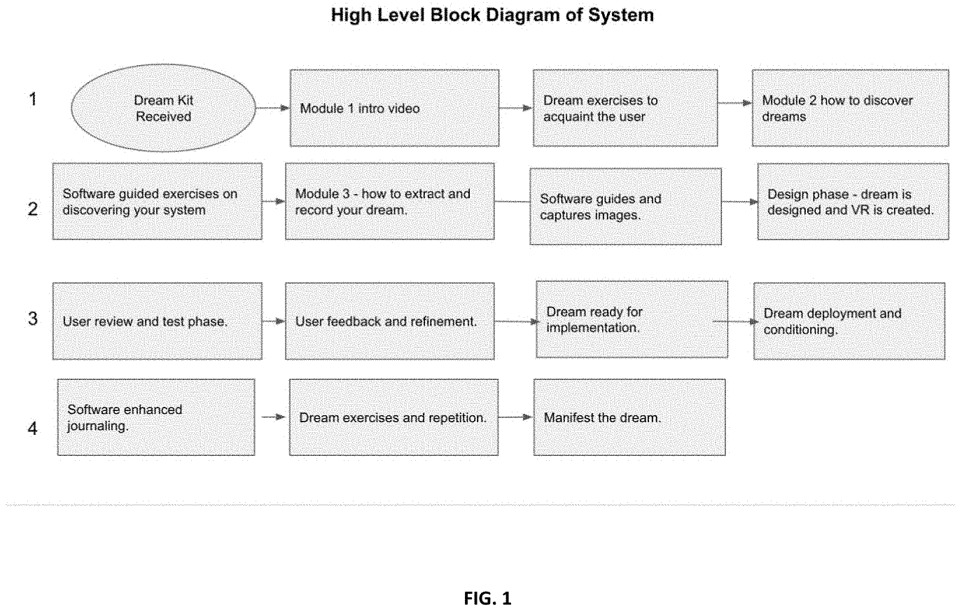

FIG. 2 is a high-level flowchart of a method according to an embodiment; and

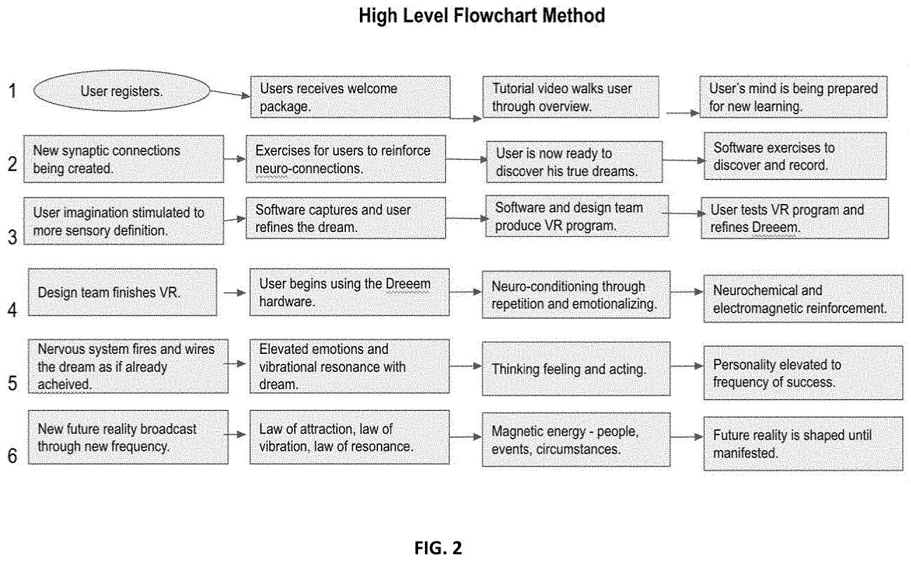

FIG. 3 high-level flowchart of a service, according to an embodiment.

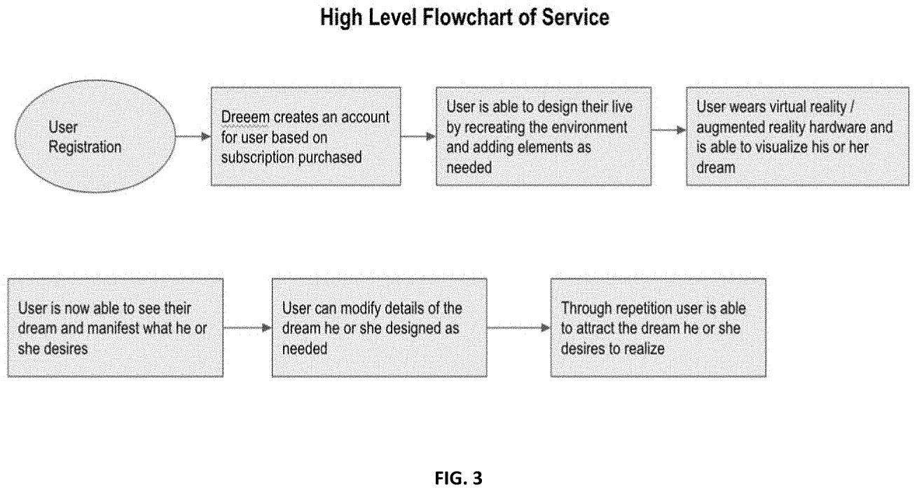

While the invention as claimed can be modified into alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the scope of the present invention.

### DETAILED DESCRIPTION

Before describing in detail embodiments that are in accordance with the present invention, it should be observed that the embodiments reside primarily in combinations of method steps and system components related to systems and methods for placing computation inside a communication network. Accordingly, the system components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Thus, it will be appreciated that for simplicity and clarity of illustration, common and well-understood elements that are useful or necessary in a commercially feasible embodiment may not be depicted in order to facilitate a less obstructed view of these various embodiments.

In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The Method

Learning is creating new synaptic connections in the brain. Thoughts cause a neurochemical to be released, called a feeling or an emotion. The moment you feel something new, and you can repeat it, you are teaching your body chemically what your mind has intellectually understood. Knowledge is for the mind and experience is for the body. Thoughts are the language of the mind and feelings are the language of the body. If you can repeat any experience over and over in your mind, you are going to neuro-chemically condition your mind and body to begin to work as one toward that experience. You are rewriting your biological program. If you repeat an experience over and over it becomes innate and automatic and it gets easy to fire and wire those nerve cells. It becomes conditioned in your nervous system and it becomes the programming, so you don't have to work at it. It is now subconsciously wired in your body. You are neuro-chemically and electro-chemically conditioning the body and mind to a certain experience and when you do it over and over you impress it onto your subconscious mind and it is now installed as a subconscious program.

Accordingly, an object of the present invention is to teach a user the philosophy and the process, provide them the easy-to-use tools to practice and condition it, and then offer the repeatability on-demand to master it until they attract and manifest their dreams. The invention takes the user from the philosophy, to an apprentice, to a master, to a self-made success. The invention takes the user from thinking about it, to doing it in their mind, to embodying it, and then to attracting it and having it or becoming it.

We each come equipped with all the biological and neurological hardware to envision and imagine our desired outcomes in order to create it by dreaming it until we manifest it. The invention taps into the latest discoveries in neuro-science and mind power, as well as the most advanced technology to teach the user how to do it brilliantly, predictably, and using the technology of virtual reality and the power and drama of cinematography to create an ultra-powerful experience of the future that will magnetize the user toward that destiny and will magnetize that destiny toward the user. The user literally begins to attract the people, events and circumstances necessary to help materialize their dream.

This highly powerful neuro-chemical process, together with it's easy to use, highly advanced technology, allows people from all walks of life to do the uncommon. The things that yesterday might be considered heroic and supernatural are now available and attainable by the regular person.

Most people struggle for years to learn the principles and process of using visualization and imagination to manifest, if they ever get to learn it. And even then, the practice is cumbersome and often ineffective because it is hard to master it to a point where you can see your dream consistently as if already happening and with enough clarity, definition, vividness and realism as to get the dreamer to feel the experience as if it was taking place. The invention bridges that gap so that the common person, not endowed with a privileged IQ or super-power, can do it in an exemplary manner with predictable quality and exactitude, on-demand, as many times as he wishes. That is the power of the present invention.

Use of the invention opens up a world of possibilities to the regular person, and even magnifies the world of possibilities for the uncommon person.

All thoughts carry a vibrational frequency and energy signature. The average person thinks 60,000 to 70,000 thoughts in one day, but most of them were not of his own creation or design. The invention helps focus the user's thoughts on that which they want and away from that which they don't want.

People emit energy and information dependent on our thoughts. Thoughts lead to choices, choices lead to behaviors, behaviors lead to experiences, experiences lead to emotions, and emotions lead to thoughts. A person's biology, neural circuitry, neuro-chemistry, and genetic expression all equal how the person thinks, acts and feels. How a person thinks, acts and feels equals that person's personality.

A person's personality creates their personal reality. If a person wants to create a new personal reality or a new life, they must change themselves or their personality. They must change or modify what they think about and how they think about it. They must elevate their vibrational frequency to the vibrational signature of the success they want to achieve. They must adopt and embody that energy before the experience. That is what the present invention enables them to do.

This inventive system and process lets a user decide what thoughts they want to put their energy behind and helps them raise their vibrational frequency to match the vibrational frequency of a person who has already achieved what they dream of, ahead of the experience.

This activity begins to install those neuro-circuits in the user's brain. And as they keep thinking that way, firing and wiring the same signature, the hardware now becomes like a software program and this newly installed software will induce them to start thinking like that great person, and that will cause them to start behaving like that great person, and feeling like that great person, and producing the results of that great person, which produces more of the thoughts of that great person, and before they know it they look in the mirror and they examine themselves they will start to see that they are now that great person.

The invention enables a user to rehearse over and over in their mind who they are going to be to experience their dream. Through the invention, they are pre-creating the experience in their mind in true color, HD, ultra-reality, with all the drama and feel of the best cinematography. When the user rehearses it vividly with the invention, neuro-chemical, electro-magnetic conditioning, their brain won't know the difference between that which they experienced inside and a real experience outside. So, they are firing and wiring the same genes and producing the same neural chemicals and neuro-circuits in their body and mind as if they already lived that very same experience. They have just pre-created their new life and are living it by experiencing it in the inventive technology on demand.

By using the invention, a user installs neural circuits in their brain, priming it, so they have the hardware and the software in place to act, attract it, and experience their dream as it manifests itself.

When the user plays and replays their dream enough times through the inventive system, they will begin to embody that person and act like that person in their waking hours, because it has been installed into their programming and has been rehearsed so many times that it becomes innate and automatic and they now just have to let their subconscious mind take the wheel. Through the invention, the user begins to teach their body and mind emotionally and neurochemically what their future is going to look and feel like before it is made manifest.

The inventive system and method described here allows a user to control their inner world to shape and mold their outer world in congruence to their dream. Use of the invention changes the old beliefs of cause and effect to the new quantum physics-enlightened belief that says you don't have to wait for something outside of you to happen to feel happy, or joy, or love, or abundance. The quantum model says you can create or pre-create an unlimited reality and it all starts with your thoughts, your imagination, and your dreams.

Science tells us that the environment and the experience signals the genes and the genes are producing the proteins, and the proteins are responsible for the structure and the function of the body. Proteins are the expression of life and genes turn on and off to dictate what neuro-chemical and neuro-circuitry to fire and wire. Therefore, when a person begins to experience the emotion ahead of the environment, the person is signaling the creation of new genes ahead of the reality and they are now pre-creating their life and their personality and, therefore, their destiny. The person's genes are literally beginning to make new proteins to be biologically and neuro-chemically prepared ahead of the experience, and they are already prepared for the future which they have just pre-created. Neurologically, neurochemically, electro-magnetically, hormonally, and emotionally, the person is being prepared ahead of their environment because they are pre-living their dream with the systems and methods of the present invention.

The body and the subconscious mind don't know the difference between a vividly imagined experience that has been created by thought alone and a real life experience. How a person thinks and feels creates their state of being.

Immune genes make immune proteins. But if you are living in anger and fear and stress and anxiety, you are down-regulating and degenerating the genes and sooner or later the degeneration of those immune genes and the up-regulating of the stress hormones will cause a condition called dis-ease (disease), and your body will respond and make the disease take form in some illness. The invention disclosed is the antithesis of this and if you focus your mind and body on pre-living the dream in a virtual, vivid experience, you up-regulate the genes and cause them to fire and wire in patterns to resemble the success of the dream ahead of the reality. The invention pre-wires the user for success by firing and wiring the success genes. You give up the story of your past and you subordinate it to the more vivid and clear story of your future by having that future fresh in your mind in HD, ultra-VR, on demand for success by firing and wiring the success genes.

We are electromagnetic beings and we emit energy and information. Just like a radio station broadcasts its signal, we broadcast a signal out to the universe consistent with our vibrational frequency. We also act as the receiving station, drawing to us energy in resonance to our own frequency. Thus, you hear of one person that has the “Midas Touch” and everything he touches turns to gold . . . . While another poor soul has a “Dark Cloud” that follows him and bad omens chase him wherever he may go. They are emitting a frequency and they undoubtedly are receiving in harmony and in resonance that which they are broadcasting out into the ether. When you are living in a stress response, you down regulate your genes and emit a very low vibrational frequency consistent with survival frequencies. This saves you vital energy for fight or flight. Normally, it is our environment that influences how we think and feel, unless we use our imagination and make that so real that we let that influence even more how we think and feel by making it so real that it surpasses is our senses.

The core of the neuro-chemical and neuro-circuitry conditioning training service disclosed here includes the method steps of:

user registers;

users receive welcome package;

tutorial video walks user through overview;

user's mind is being prepared for new learning;

new synaptic connections being created;

exercises for users to reinforce neuro-connections;

user is now ready to discover his true dreams;

software exercises to discover and record;

user imagination stimulated to more sensory definition;

software captures and user refines dream;

software and design team produce VR program;

user tests VR program and refines;

design team finishes VR;

user begins using the hardware;

neuro-conditioning through repetition and emotionalizing;

neurochemical and electromagnetic reinforcement

nervous system fires and wires the dream as if already achieved;

elevated emotions and vibrational resonance with dream;

thinking, feeling, and acting;

personality elevated to frequency of success;

new future reality broadcast through new frequency;

law of attraction, law of vibration, law of resonance;

magnetic energy—people, events, circumstances; and

future reality is shaped until manifested.

By utilizing this method, a user can harness the power of manifestation to effectuate neuro-chemical and neuro-circuitry conditioning with the goal of improving the probabilities of reaching the desired goal.

In a preferred embodiment of the invention, a user interacts with hardware systems and programmed modules to conduct the desired neuro-chemical and neuro-circuitry conditioning. The method steps comprising:

accessing by the user an account module located on a remote server via a computing device connected to a network;

validation by the account module of the user's credentials and profile data;

presenting to the user via the computing device a discovery module;

guiding by the discovery module the user to discover, extract, describe, and synthesize the user's goals and dreams into one or more inputs;

recording by the discovery module the one or more inputs into a database;

providing a design module to the user for the designing of an environment;

accessing by a design module the database to retrieve the one or more inputs to be used in designing the environment;

providing a virtual reality module;

accessing by the virtual reality module the environment and other inputs by the user;

generating by the virtual reality module a full-immersion virtual reality environment program;

storing by the virtual reality module onto a storage device the full-immersion virtual reality program;

providing a virtual reality headset hardware device;

accessing of the full-immersion virtual reality program by the virtual reality headset hardware device;

wearing by the user the virtual reality headset hardware device;

playing by the user of the full-immersion virtual reality program via the virtual reality headset hardware device;

repeating by the user of the full-immersion virtual reality program via the virtual reality headset hardware device; whereby,

the neural-chemical and neuro-circuitry conditioning training of the user is accomplished.

The Service

The neuro-chemical and neuro-circuitry conditioning training method described herein is preferably provided to users as a service (hereinafter, the “service”). Revenue models for the service may include software-as-a-service subscription models, pay-as-you-go models, or one-time fee models. Users that register and pay for the service receive software modules that provide special programming for the client-provided computing devices. Users of the service also receive visualization hardware, such as VR headsets, on a loan, lease, or purchase basis.

Registered users of the service may also receive access to specialized content, personal coaching, and other resources tailored to their neuro-chemical and neuro-circuitry conditioning neuro-chemical and neuro-circuitry conditioning training.

Upon registration the user will be asked for: (1) contact details and (2) specific details on what achieving the dream looks like for the user. The system will gather key variables of the description provided to determine a starting point in terms of background and scene to provide a starting point for the user's journey as he or she begins to design their desired dream.

The System

Embodiments of the invention include a system in which the novel neuro-chemical and neuro-circuitry conditioning training method system architecture can be implemented. The system includes a hosting zone, server, a client device, database coupled with the server, and an application gateway facilitating communication between the client device and the server. The client device can be any wireless mobile computing device such as a cell phone, tablet, personal digital assistant, and the like. The client device may also, in embodiments, be a desktop or laptop computer.

The system can be implemented as follows. A user interested in neuro-chemical and neuro-circuitry conditioning training joins the service. Joining the service can include registering, signing up, and/or or logging in. A user can log in as a registered user or “guest” user.

Once registered and signed in, the user provides some personal information that the server uses to generate a user profile, which is stored in a data store. The user profile can be updated at any time.

From a user's perspective, and in an exemplary and non-limiting embodiment, the method is implemented utilizing both a specially-programmed computing device and a VR headset. Initially, a user utilizes a user interface on the computing device to register for the training program. By way of example, and not limitation, the user may use a mobile phone connected to the internet to access a provided website. The website would prompt the user for various registration details, validate payment, and provide a way to login and create a profile.

The system software provides a series of options based on the level of subscription purchased. The software enables the user to select elements as needed to design an environment including the background, landscape, colors, family members, friends, co-workers, vehicles, clothing and any other element the user wishes to see while being able to visualize with vividness their desired dream.

Once registered and logged-in, the user is presented with instructions guiding the user to discover, explore, and define their goals for the neuro-chemical and neuro-circuitry conditioning training. In embodiments, the user may be presented with an introduction video or other audio visual presentation explaining how your imagination works to create your reality.

Software modules provide a user interface to guide the user in the creation of assets to be used in the visualization of their goals.

The software educates the user on what a real or worthy goal is or what a worthy ideal looks like and more importantly what it feels like. This is important because a lot of people dream of something only because their mother wanted them to be a doctor or a lawyer, etc. it is vital to help the user uncover their real dream and let them feel the difference from someone else's dream. Only your true dream will end up fulfilling you. The power of the dream is in who you will become. Once the user understands what a real dream looks like and feels like, the software will guide him using a closed-eye process (where the user closes his eyes and is guided to imagine their dream and picture it), to dig deep and discover their true dream and then to give it more feeling and sensory responses.

The software will prompt the user following the closed-eye process to record and document the dream in a very descriptive manner and provoke more and more definition until it is imbedded and also descriptive enough to allow a very vivid VR model to be produced. The software input feeds into a design model and a VR program is produced. The VR program is then reviewed and edited by the user and the design team.

Once finishing touches are put on it and the user experience is such that it fully associates him with actually living that experience, then the program is ready to be exercised and put to use on demand and repeated at regular intervals and increasing frequencies until the people, events, and circumstances are attracted and the dream is manifested.

Once a virtual reality environment is created based upon user inputs, the VR programmed environment is loaded via the computer network to the VR headset. The programmed environment may also be stored on a memory device (such as an SD card) and loaded locally onto the VR headset.

The system described above is only one example of a suitable system and is not intended to limit the scope of use or functionality of embodiments of the present invention described above. The system is operational with numerous other general purpose or special purpose computing system environments or configurations. Examples of well-known computing systems, environments, and/or configurations that may be suitable for use with the information processing system include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, clusters, and distributed cloud computing environments that include any of the above systems or devices, and the like. Once programmed to perform the method steps utilizing the modules described in the invention disclosed, general purpose computing systems become specially-programmed to perform said method steps.

The system may be described in the general context of computer-executable instructions, being executed by a computer system. The system may be practiced in various computing environments such as conventional and distributed computing environments where tasks are performed by remote processing devices that are linked through a communications network. In a distributed computing environment, program modules may be located in both local and remote computer system storage media including memory storage devices.

The system is comprised of client-side and server-side hardware components that communicate with each other over a data network in order to execute the method steps, according to an embodiment of the disclosure.

The client device may represent any type of mobile programmable electronic device, such as a smartphone, wearable computing device such as the Apple Watch®, tablet computer, personal digital assistant, laptop computer, and the like. The client device can include a processing device coupled with memory, storage, and in-device features such as GPS, all operatively linked by bus. One with knowledge in the art will appreciate that other components will be included in order to operate a commercially feasible device; however these are not depicted herein for simplicity and clarity of illustration.

The client device, in its embodiment as a mobile device, includes a Mobile Operating System (OS) which can be a proprietary OS developed and sold by a provider, such as iOS developed by Apple Inc., and Android OS developed by Google. The Mobile OS contains operating system features, in addition to other features common to smartphones, such as GPS, camera, music player, Bluetooth, and the like.

The Mobile OS will also include various apps, some of which are supplied “out of the box” (included with the device), and some of which are later acquired by the user. The apps can include, for example, XMPP, GPS, and the like. Future marketing and development will decide in which manner the service app will be provided. The User Interface App is what determines how the user interacts with the device. The User Interface App determines the “look and feel” of the various apps and controls such things as touch screens, swipes, icon views, and the like.

The Server is a computing system which can be a stand-alone computer or networked into a larger system. For purposes of this disclosure, the Server is a networked computing device in communication with other network devices. It will be readily appreciated that the Server, as a computer, includes conventional hardware and functionality required to operate as a provider of the service. This hardware and functionality are well known to those with knowledge in the art and thus are simply represented in the diagram as Server Processing/Storage components.

The User Interface Engine provides the service's graphical user interface with which the user of the service engages. Interaction between the user and the service is directed by the User Interface Engine, such as providing screens, receiving input, providing pop-up windows, and the like. The user interface processing is preferably performed using technologies such as Java-based graphical user interface technologies, although other similar technologies now known or subsequently developed may be used for the processing.

The Register Engine handles the user registration and interfaces with the Monetization Engine for processing any fees or upgrades attached to the registration. The Register Engine also builds the user profile.

The various modules and steps described in the method, above, are preferably embodied as modules or “engines” of the system. For example, the Discovery Engine handles the querying and exploration of the user's goals. The Creation Engine handles the designing of assets and environments. The Visualization Engine handles the “playback” of the user's created full-immersion environment.

### Alternative Embodiments

As the full-immersion visualizations are digitally created virtual reality environments, the experience could be more and more detailed and include enhanced reality experiences, which could include their family members being in the VR set, their actual house, car, accessories and it could have almost infinite levels of possibilities.

While discussed here in the context of dreams and goals, the present invention could also be used for people to promote self-healing. This embodiment could have added modules or options, where the user is able to, for example, “feel” their legs using additional hardware such as neuro sensors or electrodes that stimulate that portion of the brain that causes the sensation of feeling their legs again.

In the preferred embodiment described above, the hardware implemented is VR goggles, however, the solution could integrate with other software and hardware technologies, such as televisions, projection screens, body suits with sensors and haptic feedback, or a simulation booth where the user can experience not only the visual component of the solution but also he or she can experience the full-immersion environment in a more detailed and vivid quality stimulating the five senses: visual, auditory, kinesthetic, olfactory and gustatory.

An example of the above could be where someone in a wheelchair and with limited or no use of their legs could put on the hardware and see himself pushing up on the arms of the wheelchair and see themselves rising up and look down to watch how his legs are now supporting him. He could further see his legs moving and taking steps and walking. He could raise his eyes and see himself at eye-level with the bathroom mirror that he hasn't been able to see himself in for quite some time. He could look at his reflection on the bathroom mirror and see his own look of amazement and joy. So this image could get fired and wired in his nervous system and anchored in his subconscious so that the doubt he has harbored of ever being able to walk quickly dissipates and makes room for faith and expectation that it is just a matter of time. His ability to see it with this HD vividness and in Ultra-VR technology with the latest in quality and sensory developments now allows him to raise his vibrational frequency and create a very dramatic change in his neuro-chemistry and neuro-circuitry that prepares his body and mind for self-healing and a much swifter recovery. The invention allows him to create his own placebo effect each time he puts on his VR goggles and experiences the healing taking place with his very own eyes.

In light of the foregoing description, it should be recognized that embodiments in accordance with the present invention can be realized in numerous configurations contemplated to be within the scope and spirit of the claims. Additionally, the description above is intended by way of example only and is not intended to limit the present invention in any way, except as set forth in the claims. Modifications and changes to the invention should be readily apparent to those having ordinary skill in the art, which modifications are intended to be within the spirit and scope of the invention as claimed. It is also understood that the foregoing description is illustrative of the present invention and should not be considered as limiting. Components from one embodiment can be combined with another embodiment and remain within the spirit and scope of the invention. Therefore, other embodiments of the present invention are possible without departing from the spirit and scope of the present invention.

1. A method for neuro-chemical and neuro-circuitry conditioning and training, the method comprising the steps of:
registering by a user;
receiving by the user of a welcome package;
providing a tutorial video whereby the user is walked through an overview;
preparing of the user's mind for new learning;
creating in the user new synaptic connections;
reinforcing of the user's neuro-connections through performing of exercises;
discovering by the user of their true dreams;
providing of software specially programmed to run the user through exercises configured to discover and record the user's dreams;
stimulating by the software of the user imagination thereby creating more sensory definition;
capturing of the user's dream by the software;
refining of the user's dream;
producing of VR program by the software;
providing VR program to user for testing and refining;
providing to the user a finished VR experience;
providing to the user specially programmed hardware configured to present the VR experience to the user; and
using of the specially programmed hardware by the user configured such that the user experiences neuro-conditioning through repetition and emotionalizing, as well as, neurochemical and electromagnetic reinforcement causing the nervous system to fire and wire the dream as if already achieved, and creation of elevated emotions and vibrational resonance with the dream through thinking, feeling, and acting causing the user's personality to be elevated to the frequency of success and a new future reality is broadcast through the new frequency through the law of attraction, the law of vibration, the law of resonance, and through magnetic energy people, events, circumstances and the future reality is shaped until manifested,
whereby, through the method, the user harnesses the power of manifestation to effectuate neuro-chemical and neuro-circuitry conditioning with the goal of improving the probabilities of reaching the desired goal.

2. A method adapted such that a user interacts with hardware systems and programmed modules to conduct a desired neuro-chemical and neuro-circuitry conditioning, the method comprising the steps of:
accessing by the user an account module located on a remote server via a computing device connected to a network;
validation by the account module of the user's credentials and profile data;
presenting to the user via the computing device a discovery module;
guiding by the discovery module the user to discover, extract, describe, and synthesize the user's goals and dreams into one or more inputs;
recording by the discovery module the one or more inputs into a database;
providing a design module to the user for the designing of an environment;
accessing by a design module the database to retrieve the one or more inputs to be used in designing the environment;
providing a virtual reality module;
accessing by the virtual reality module the environment and other inputs by the user;
generating by the virtual reality module a full-immersion virtual reality environment program;
storing by the virtual reality module onto a storage device the full-immersion virtual reality program;
providing a virtual reality headset hardware device;
accessing of the full-immersion virtual reality program by the virtual reality headset hardware device;
wearing by the user the virtual reality headset hardware device;
playing by the user of the full-immersion virtual reality program via the virtual reality headset hardware device;
repeating by the user of the full-immersion virtual reality program via the virtual reality headset hardware device; whereby,
the neural-chemical and neuro-circuitry conditioning training of the user is accomplished.

3. A system adapted to implement the neuro-chemical and neuro-circuitry conditioning training method of claim 2, the system comprising:
a hosting zone;
a server;
a client device;
a database operationally coupled to the server;
an application gateway configured to facilitate communication between the client device and the server;
a specially programmed computing device in operational communication with a VR headset;
a user interface engine configured to perform the steps of:
accessing by the user an account module located on the server via the client device connected to a network;
validation by the account module of the user's credentials and profile data;
and presenting to the user via the computing device a discovery module;
providing a design module to the user for the designing of an environment; and
providing a virtual reality module;
a register engine;
a monetization engine;
a discovery engine configured to perform the steps of:
guiding by the discovery module the user to discover, extract, describe, and synthesize the user's goals and dreams into one or more inputs; and
recording by the discovery module the one or more inputs into a database;
a creation engine configured to perform the steps of:
accessing by a design module the database to retrieve the one or more inputs to be used in designing the environment;
accessing by the virtual reality module the environment and other inputs by the user;
generating by the virtual reality module a full-immersion virtual reality environment program; and
storing by the virtual reality module onto a storage device the full-immersion virtual reality program;
and
a visualization engine configured to perform the steps of:
accessing of the full-immersion virtual reality program by the virtual reality headset hardware device;
wearing by the user the virtual reality headset hardware device;
playing by the user of the full-immersion virtual reality program via the virtual reality headset hardware device; and
repeating by the user of the full-immersion virtual reality program via the virtual reality headset hardware device.
